(19) United States
(12) Patent Application Publication
(10) Pub. No.: US 2024/0065739 A1
(43) Pub. Date: Feb. 29, 2024

(54) RESCUE EXTERNAL FIXATION SYSTEM

(71) Applicant: William MONTROSS, Colorado Springs, CO (US)

(72) Inventor: William MONTROSS, Colorado Springs, CO (US)

(21) Appl. No.: 18/387,389

(22) Filed: Nov. 6, 2023

Related U.S. Application Data
(63) Continuation-in-part of application No. 17/685,086, filed on Mar. 2, 2022.
(60) Provisional application No. 63/155,673, filed on Mar. 2, 2021.
(60) Provisional application No. 63/422,809, filed on Nov. 4, 2022.

(51) Int. Cl. A61B 17/80; A61B 17/66

(52) U.S. Cl. CPC A61B 17/8047; A61B 17/66; A61B 17/8052

(57) ABSTRACT

Disclosed are improved devices, systems and methods for attaching a rod or other connector(s) to an external fixation frame or other object.

### CROSS-REFERENCE TO RELATED APPLICATIONS

This application is a continuation-in-part of U.S. patent application Ser. No. 17/685,086 entitled “CONNECTION DEVICE,” filed Mar. 2, 2022, which claims the benefit of U.S. Provisional Patent Application Ser. No. 63/155,673 entitled “HOOK NUT,” filed Mar. 2, 2021, the disclosures of which are hereby incorporated by reference in their entireties.

This application further claims priority to and the benefit of U.S. Provisional Patent Application Ser. No. 63/422,809 entitled “RESCUE EXTERNAL FIXATION SYSTEM,” filed Nov. 4, 2022, the disclosure of which is hereby incorporated by reference in its entirety.

### TECHNICAL FIELD

The invention relates to improved devices, systems and methods for attaching rods and/or other connector(s) to an external fixation frame or other object. More specifically, embodiments disclosed herein can facilitate creation of support and/or fixation frame structures or components thereof that can easily be particularized for the needs of an individual patient, including the incorporation of various removeable and/or configurable components of the frame that allow the frame to be reconfigured in a variety of ways without requiring removal from and/or loss of support to the patient.

### BACKGROUND OF THE INVENTION

Reduction is a medical procedure to restore a fracture or dislocation to the correct alignment. When a bone fractures, the fragments lose their alignment in the form of displacement or angulation. For the fractured bone to heal without substantial deformity the bony fragments must be re-aligned to their normal anatomical position. Orthopedic surgeons attempt to recreate the normal anatomy of the fractured bone by reduction.

Fractured bone reduction or treatment can include use of fixation methods that can reinforce the fractured bone and keep it aligned during healing, including use of external devices or casts as well as internal devices such as rods, bone plates and/or fasteners. Under certain circumstances, a physician may decide that external fixation is the best treatment for a patient. Fixation with external devices and assemblies includes surgical techniques for setting bone fractures and/or for limb lengthening that was first used more than a century ago. Since that time, the technique has evolved from being used primarily as a last resort fixation method to becoming a main-stream technique used to treat a myriad of bone and soft tissue pathologies.

In some cases, external fixation can be accomplished by placing pins or screws into the bone of a patient and securing the pins through the use of an external frame assembly positioned at least partially outside the body. During the treatment, the external frame can hold bone fragments at adjustable spacing and angles to create a desired overall bone length and angular disposition of the bone fragments. To connect the external fixation device to the bone, pins can be placed, for example, on either side of the break in the bone and pass through the skin and sometimes the muscles. Sometimes wires can also be used with the pins, or in place of pins, to secure the bone pieces. The pins and/or wires can hold the bone in place and anchor the fixator securely, while also avoiding damage to vital structures, allowing access to the area of injury, and meeting the mechanical demands of the patient and the injury. Treatment using external fixation can take about 6 weeks for a simple fracture, and up to one year or longer for a more complicated fracture.

As compared to other fixation methods, external fixation devices can provide numerous advantages. When compared with internal plates and intramedullary nails, for example, external fixators can cause less disruption of the soft tissues, osseous blood supply, and periosteum. Accordingly, external fixation devices can be useful for soft tissue management in the setting of acute trauma with skin contusions and open wounds, in chronic trauma where the extremity is covered in thin skin grafts and muscle flaps, and in patients with poor skin whose healing potential is compromised as in the case of rheumatoid disease, peripheral vascular disease, diabetes mellitus, and Charcot disease. In addition, the temporary nature of the pins and wires can provide bony stability in the setting of osteomyelitis where the presence of internal implants make eradication of infection more challenging. The ability to avoid putting fixation into the infected area is equally beneficial.

Unlike internal plates and intramedullary nails, external fixators also provide postoperative adjustability. This allows the extremity to be manipulated in the operating room to gain exposures to fracture fragments. In the situation of limb lengthening or deformity correction, gradual manipulation is possible with frame adjustment over time. As a result, external fixations have found use in pediatric fracture care where open physes preclude intramedullary nailing. Leg length discrepancy can also be reliably treated with circular and monolateral design fixators.

Many different designs and arrangements of fixation frames are known in the art, but many of the existing designs require a multiplicity of rods or bars interconnecting pin-holders in the frames, and the complexity of interconnecting frame structures can interfere with surgical management during placement of the frames and adjustment of the bone segments interconnected by the frames. In particular, room for improvement exists in providing a lightweight fixation frame that can be particularized for the needs of an individual patient, including the incorporation of various removeable and/or configurable components of the frame that allow the frame to be reconfigured in a variety of ways without requiring removal of the frame from the patient, and which performs similar or superior to existing fixation frames and/or that can be produced at a reasonable cost. Thus, there is continued interest in providing improved external fixation devices that are more versatile and can be used, for example, as more ambulatory or portable devices.

### BRIEF SUMMARY OF THE INVENTION

The present invention includes the realization of a need for improved attachment mechanisms and associated structures which obviate many of the difficulties characterizing prior known fixation frame components. In various embodiments, the devices and systems disclosed herein can facilitate attachment, removal and/or adjustment of a support and/or fixation frame, including components that allow the frame to be reconfigured in a variety of ways without significantly affecting the strength and/or stability of the frame or requiring removal of significant frame structure from the patient.

The present invention relates to orthopedic fixation systems, assemblies, devices and related methods for reduction of a fractured bone of a patient.

Various embodiments disclosed herein include clamping or connecting devices for use with an external fixation frame system. The disclosed clamping and/or connecting devices can include a fixed jaw or gripping section and one or more adjustable compressor or post component which can be extended towards and/or away from the fixed jaw/gripping section. In various alternative embodiments, the fixed jaw or gripping section can include one or more a concave interior surface(s).

In various embodiments, a method of building an external fixation system for stabilizing and reducing a bone is described. The method can include arranging one or more of the disclosed clamping devices to be utilized in the construction of the external fixation system and/or during modification thereof.

In various embodiments, a wide variety of shapes, sizes and/or functions can be integrated into the fixed and/or adjustable components of the clamping device, and such devices can desirably be utilized with a wide variety of shaped support structures, including arched, square and/or rectangular support plates or blocks, which can be interconnected using a variety of rods and connectors. Desirably, the support blocks can include openings and/or other connection features that allow connecting rods to be attached thereto, including at various angles. The support rods can comprise straight rod supports as well as angled and/or curved support rods. The disclosed systems can desirably facilitate the use of different sized and/or shaped support rods and/or support blocks on a single patient, which can be particularly useful for treating patients who are highly muscularized or are extremely obese or those with atypical anatomical features. In many embodiments, the systems disclosed herein can provide an extremely strong, durable and/or rugged construct which can be configured to easily accommodate virtually any patient and/or anatomical situation, and which can be reconfigured without requiring removal from and/or loss of support to the patient's anatomy.

Other important features of the various designs disclosed herein facilitate the quick and convenient assembly, disassembly and/or repositioning of various frame components, which can facilitate faster access to various underlying anatomical regions to allow dressing changes and/or alteration of frame components, including an ability to change “levels” in an office and/or outpatient visit.

In various embodiments. the frame configuration can be particularly quick and easy to assemble and/or disassemble in a much more rapid fashion than with existing fixation frame designs. Moreover, the disclosed components can easily accommodate multiple different sizes, shapes and/or designs of products produced and/or sold by different manufactures, which heretofore has been difficult to accomplish in a quick, easy and cost-effective manner.

In various embodiments, frame components can utilized to quickly and easily “upsize” and/or “downsize” some portions and/or all of the frame at various anatomical locations on the patient. For example, an athletic and/or obese individual may have various enlarged anatomical features such as enlarged calves and/or thighs, with more normally sized and/or shaped feet and ankles. For these individuals, it may be desirous to assemble a modular frame having enlarged components in the calf or thigh region, while retaining more normally sized and/or shaped components in the foot and/or ankle region. In addition, the various disclosed embodiments can optionally include components can be shifted, rotated and/or tilted relative to other components in the frame, allowing a modular frame design to be constructed that accommodates enlarged and/or unusually shaped anatomy in various regions, as well as anatomy that may be disposed in a significantly lateral direction relative to the longitudinal axis of the frame.

The various modular frame components disclosed herein allow for removal and/or reconfiguration of individual frame components, most desirably without requiring removal and/or reconfiguration of other adjacent components. Unlike existing frame systems that typically incorporate multiple nuts and/or other attachment mechanisms on inner walls and/or other difficult/impossible to access locations of the frame, the connection devices, frames and related components include externally accessible connection features that can be easily accessed while being worn by the patient, allowing for the quick and convenient addition and/or removal of frame sections and/or components at virtually any location, including during out-patient office visits.

### BRIEF DESCRIPTION OF THE DRAWINGS

The drawings and the following description relate to preferred embodiments by way of illustration only. It should be noted that from the following description, alternative embodiments of the components and methods disclosed herein will be readily recognizable as viable alternatives that may be employed in one skilled in the art.

FIG. 1A depicts an exemplary PRIOR ART fixation frame positioned around a leg bone of a patient;

FIG. 1B depicts another exemplary PRIOR ART fixation frame positioned around a leg bone of a patient;

FIG. 1C depicts another exemplary PRIOR ART fixation frame construct utilizing multiple compression nuts which secure a support rod to a frame section;

FIGS. 2A and 2B depict perspective views of one exemplary embodiment of a clamping or connecting device;

FIG. 2C depicts a perspective view of a set screw that can be utilized with the connecting device of FIGS. 2A and 2B;

FIGS. 3A and 3B depict perspective views of another exemplary embodiment of a clamping device connecting a threaded or unthreaded support rod or strut to a plate member;

FIG. 3C depicts a perspective view of another exemplary embodiment of a clamping device connecting a threaded or unthreaded support rod or strut to a plate member

FIGS. 4A and 4B depict front and side plan view of one exemplary embodiment of a connection device;

FIG. 4C depicts a side view of an exemplary compression bolt which can be utilized with the connection device of FIGS. 4A and 4B;

FIGS. 5A and 5B depict side and front views of one exemplary embodiment of a clamping device with various representative dimensional features;

FIGS. 5C and 5D depict side and front views of another exemplary embodiment of a clamping device with various representative dimensional features;

FIGS. 6A through 6D depict various views of exemplary dimensions that may be altered and/or accommodated in various embodiments of a clamping device;

FIGS. 7A through 7C depict plan, bottom and side views of another exemplary embodiment of a fixation plate;

FIGS. 8A through 8E depict various views of another exemplary embodiment of a clamping device;

FIG. 9 depicts the clamping device of FIGS. 8A through 8E connecting to a toothed support ring and a threaded or unthreaded support rod;

FIGS. 10A through 10F depict various views of another exemplary embodiment of a clamping device;

FIG. 11 depicts the clamping device of FIGS. 10A through 10F connecting to a curved support ring;

FIGS. 12A through 12F depict various views of another exemplary embodiment of a clamping device; and

FIGS. 13A and 13B depict a pair of the clamping devices of FIGS. 8A through 8E connecting to a curved support ring and a pair of threaded or unthreaded support rods, where the clamping devices and support ring can be selectively repositioned on the rods.

### DETAILED DESCRIPTION OF THE INVENTION

The disclosures of the various embodiments described herein are provided with sufficient specificity to meet statutory requirements, but these descriptions are not necessarily intended to limit the scope of the claims. The claimed subject matter may be embodied in a wide variety of other ways, may include different steps or elements, and may be used in conjunction with other technologies, including past, present and/or future developments. The descriptions provided herein should not be interpreted as implying any particular order or arrangement among or between various steps or elements except when the order of individual steps or arrangement of elements is explicitly described.

Orthopedic fixation systems as described herein generally include frame structures that surround and/or extend along one or more bones to allow for stabilization of a fracture and/or reconstruction of bones and/or surrounding tissue. The systems of the present invention can include a variety of components that can be selected for a desired level of stabilization. Systems of the present invention can include at least two main components: one or more support plates and interlocking spacer and support rods. As indicated herein, a support plate will generally extend around a region of the targeted anatomy, often extending transverse and/or angled relative to the anatomy, while the spacer and support rods typically extend parallel or along the anatomy. In various embodiments, bone-interface components (fixation pins, for example) that can assist in stabilizing a bone (e.g., tibia, fibula, femur or humerus), can be attached to the plates and/or rods in a variety of ways. As part of the embodiments disclosed herein, various frame components are disclosed, including plates, rings, struts, rails, and/or braces, as well as bone-interface components such as various types of orthopedic pins, rods, screws, shafts, wires, and the like that can connect to a bone, e.g., between a frame component and the patient's bone/fragment for positioning or reduction as described. Bone-interface components are commonly coupled to frame ring structures and can be coupled to various frame components, including plates, rings, struts, rails, arms, etc. The various frame components, for example, can be used by a healthcare provider to assemble the systems in a fashion to allow for varied levels of stabilization.

For use, the systems of the present invention can be generally used for medical procedures that involve fixation and/or reduction of a patient's bone, including limb stabilization. The orthopedic fixation systems of the present invention can be applied to treat various bones or fractures, including bones/fractures of both upper and/or lower limbs, such as a bone in the leg or the arm. A leg bone can include a femur, a tibia, a fibula, or a combination thereof. An arm bone can include a humerus, a radius, an ulna, or a combination thereof. In some embodiments, a segment of a bone can be treated using a device of the present invention. In certain embodiments, the orthopedic fixation systems of the present invention can also serve as reduction devices for a fractured or dislocated bone. For example, the systems can be configured to provide open or closed reduction. For open reduction, bone fragments are exposed surgically to assist in restoring a fracture or dislocation. Closed reduction can manipulate the bone fragments without surgical exposure.

In certain notable aspects, the various components and relationships therebetween can allow for increased portability and flexibility for stabilizing a bone, for example, in an outpatient setting and/or at the scene of an accident or other environments in which the ability to quickly assembly and/or easily modify the frame design can be particularly useful. The systems described herein can also provide a greater versatility in use because the systems can be assembled and/or modified to allow for different levels of stabilization of the bone. For example, in some situations, healthcare providers may desire more adjustable systems for mobility that can later be modified to increase stability upon arrival to a location that allows for such modifications, such as where an initial portability may be desired to keep the bone stabile before and/or during surgery in an operating room. As described herein, the disclosed embodiments can be assembled to include frame components that may allow for fixation or reduction of a bone in situations that involve more temporary, mobile stabilization or other situations that desirably provide greater stability that may be desired for some operating room situations, including components that may allow the fixation frame to be secured to a bed, a surgical table and/or some other support.

As described herein, the disclosed systems can include a multitude of frame components, such as plates (also commonly referred to as “rings”) and spacer/support rods. The plates can be of any size and/or shape suitable for use with the systems, devices, and methods of the present invention. The plates can include full plates and/or partial plates, such as half or three-quarter plates, straight and/or curved sections or portions thereof, and/or angular C or U-shaped plates (as well as curved equivalents thereof, if desired). Various plates will desirably further include a variety of mechanisms for mounting the spacer/support rods and/or other bone-interface components. For example, plates can include holes (including internally threaded holes and/or hole portions) ribs, indentations, depressions, protrusions and/or other features that can be coupled with spacer/support rods and/or additional mounting components, such as brackets or other structures that allow for coupling with connectors to interface with bone. In certain embodiments, the plates can be removably and/or slidably coupled to allow removal and/or horizontal displacement of the plates in relation to a bone and/or other portions of the frame structures without greatly affecting the strength and/or support to the bone provide by the remaining frame elements. Once in a desired position, the plate and/or other frame components can be tightened and/or resecured to other frame elements in a desired new position and/or orientation.

In various embodiments, the spacer/support rods or struts used in the present invention may have any suitable dimension of size or shape to, for example, provide for stabilization and/or mounting of various bone-interface components or other frame components. Rods or struts can be elongate and substantially linear in shape or a whole or part of the rod/strut can be bent (e.g., angular and/or curved). In some embodiments, a strut can be a member of a set of struts, in which each strut can be the same size/shape or of different sizes/shapes. The set of struts can include struts of the same and/or different diameter, the same and/or different maximum (and/or minimum) length, and/or the same and/or different angular adjustability. Distinct struts, of the same or different size and/or adjustability, can be marked as distinct. For example, the struts may include indicia, such as alphanumeric characters, distinct colors, removable (or permanent) colored bands, etc. In some embodiments, the indicia can be used by a healthcare professional to choose specific struts having a desired shape and/or stiffness for a particular stabilization procedure. In certain embodiments, struts can also include one or more movable joints that can, e.g., permit relative (internal) translational or pivotal motion of portions the strut. In some embodiments, the joint can allow a twisting motion about an axis parallel to a long axis defined by the strut. In addition, a joint can also permit a bending motion(s) about an axis (or axes) transverse to the long axis of the strut. The joint may be a hinge joint, a ball-and-socket joint, and/or a combination thereof, among others.

In various disclosed embodiments, at least some of the rods and/or struts can be secured to plates and/or other fixation frame components using the clamping or connecting devices described in the present invention. For example, a strut can be fastened at several points along a set of plates. Alternatively, one strut can be coupled at one end to one plate and at another other end to a second plate. The locations and orientations of the struts in relation to plates, or other components, can be dependent on the particular application of the struts for stabilizing the bone in an orthopedic fixation system of the present invention.

If desired, additional support components, such as braces, can be coupled to the various fixation frame components so as to increase or decrease the stabilization level of the orthopedic fixation systems. In certain embodiments, the braces can provide additional stabilization support as well as to provide adjustment capability for a user. Suitable brace components can include rod supports, hinges, adjustment handles, joints, etc. The braces can have a configuration that can be adjusted in a variety of ways, such as in length, angle, height, etc. In some embodiments, the braces can include at least one joint or hinge to permit internal relative motion among various components of the brace. Other components, such as adjustment handles, can be configured to allow a healthcare provider to adjust the size and/or shape of the brace as well as the way the brace can couple with other system components.

In general, the frame components and/or bone-interface components can be coupled (e.g., permanently or removably coupled) to other components through a variety of ways. The coupling mechanisms for the systems of the present invention can generally include coupling mechanisms, such as fasteners, screws, nuts, brackets, and/or bolts, as well as other ways to attach various components, such as welding, gluing, tying, etc. In addition, plates can be removably and slidably coupled to various frame components. Fixing pins and/or wires can be independently and/or removably coupled to the plates and/or rods. Alternatively, the fixing pins and/or wires can be independently and/or removably coupled to other components that are coupled to the plates and/or rods. Coupling additional components to various parts of the assembly can depend on several factors, such as the bone needed fixation and/or reduction or, e.g., the placement of a fracture in the bone.

Disclosed herein are a variety of new devices to facilitate external fixation. Various embodiments include systems of clamps, rings, bars and/or brackets (and optionally other components) that can attach to an already assembled and attached external ring fixation systems using struts that are telescoping and variable angle. Where the rings attached to the struts may or may not be inline, congruent or symmetrical. It may also comprise a hybrid system comprising traditional fixation components with various of those described herein.

The various clamps and/or rings described herein can include unique components which can attach to non-parallel struts to add a layer of protection to the already assembled and attach external fixation system (AAEF). Accessory clamps will desirably be able to hold half pins, wires or other apparatus to aid in the reduction of a fracture or alignment of the bones being held together. Clamps will be able to attach a ring, bracket or bar uniquely to struts. Accessory clams will be able to hold skinny pin, half pin, reduction bars or rods, and other apparatus needed to reduce, protect or stabilize a fracture or appendage. Use of various system components can include the act of using unique clamps on a perpendicular to non-perpendicular ring construct of variable angle struts and partial rings and bars used in surgery to attach a ring and accessory clamps. The components of this system will desirably be able to fine tune fracture reduction, extremity reconstruction, protect patient vital areas (flaps, wounds, etc.) using existing technology, including combinations of existing systems and/or uses with new technologies.

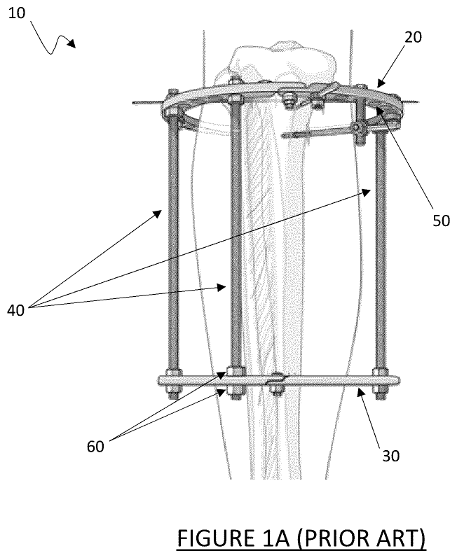

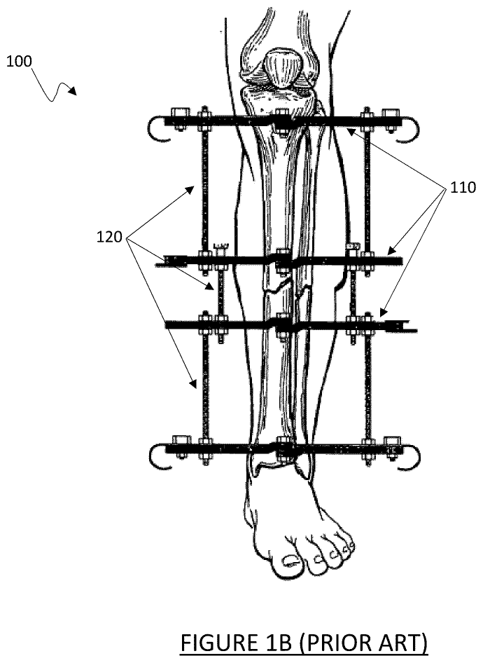

FIG. 1A depicts a prior art fixation frame 10 comprising a first fixation ring 20 and a second fixation ring 30, each of the rings 20 and 30 formed from c-shaped elements. A plurality of threaded rods or struts 40 are connected between each of the rings 20 and 30, with the rods 40 extending through openings 50 in each of the rings, with compression nuts 60 threaded onto each of the rods and positioned on each side of the respective fixation rings. While this arrangement creates a very durable and strong construct, any modification and/or removal of a ring or related c-shaped element from this construct would necessitate significant disassembly of the construct.

FIG. 1B depicts a prior art fixation frame 100 comprising a plurality of ring members 110, each member connected to adjacent members by struts 120. As in the previous construct, the struts are secured to the ring members using compression nuts on the threaded rods, with the nuts positioned on each side of each ring member to compress the ring member therebetween. In addition, the struts of this construct do not extend the full length of the construct, but rather “skip” each level to different radial positions to avoid having a single strut extend the entire length of the construct—thus allowing individual levels to be disassembled or modified without requiring disassembly and/or removal of the entire construct. However, this arrangement adds significant complexity and/or weight to construct, which is undesirable in many circumstances.

FIG. 1C depicts an exemplary prior art arrangement of compressions nuts 150 and a threaded support strut 160 which is secured to a frame member 170, alongside another unthreaded support strut 180. In this prior art arrangement, the nuts are threaded and spun onto the rod, and if the user discovers a nut is missing from the construct after even partial assembly, the assembly typically has to be disassembled. Moreover, if there is a nut missing from a three-plate or larger assembly, the user must generally at least partially remove the rod to replace the missing nut(s).

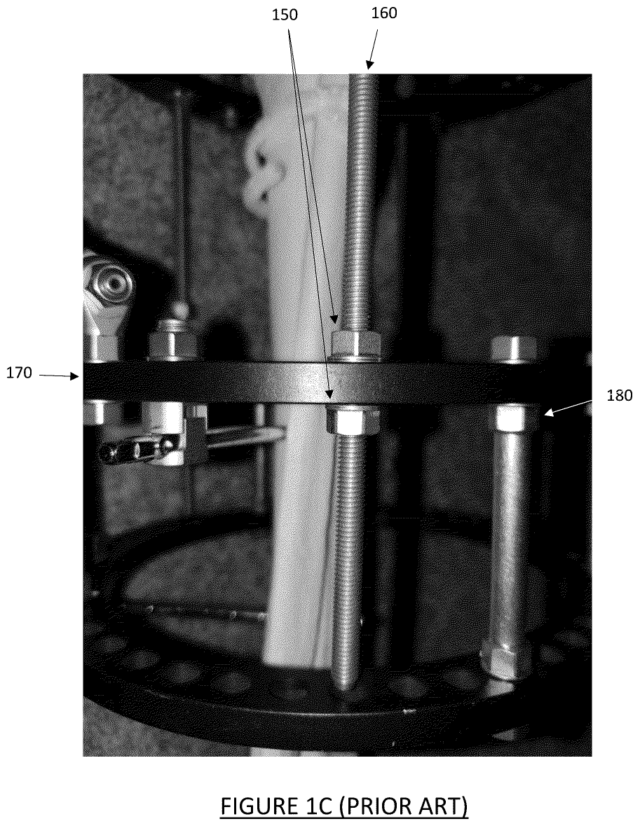

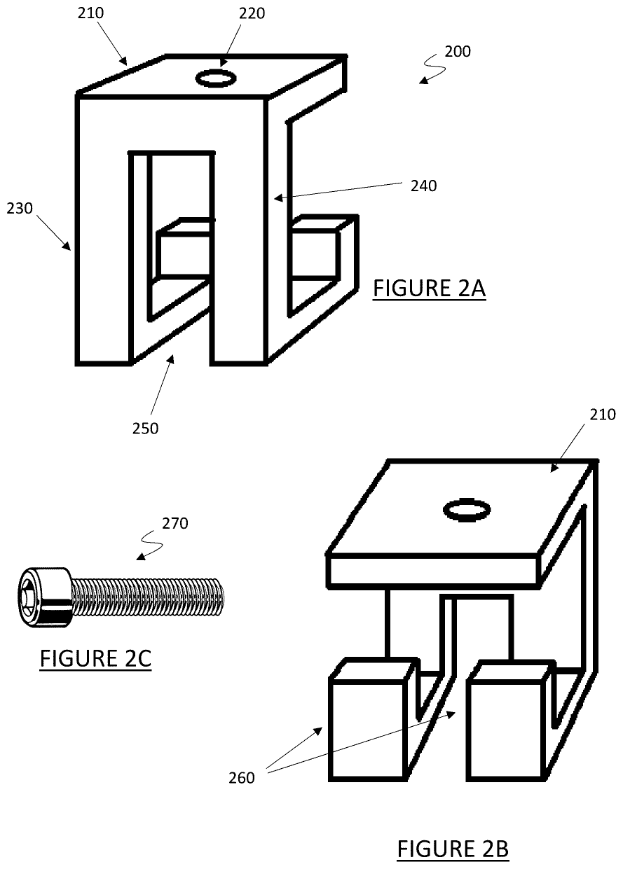

FIGS. 2A and 2B depict perspective views of one exemplary embodiment of a clamping or connecting device 200 constructed in accordance with various teachings of the present invention. In this embodiment, the clamping device 200 includes a base plate 210 having a threaded hole 220 formed therethrough, and a pair of arms 230 and 240 which extend from their proximal ends downward from an edge of the base plate 210. The arms 230 and 240 are desirably separated by a notched section 250, with each arm having a retention portion or hook end 260 positioned at a distal end thereof. An externally threaded set or compression screw 270 (see FIG. 2C) can be threaded into and at least partially through the threaded hole 220, with a distal end thereof positioned below the base plate 210. If desired, a longitudinal axis of the compression screw may be positioned parallel to at least a portion of arms 230 and 240. FIG. 2C depicts a perspective view of a set screw that can be utilized with the connecting device of FIGS. 2A and 2B.

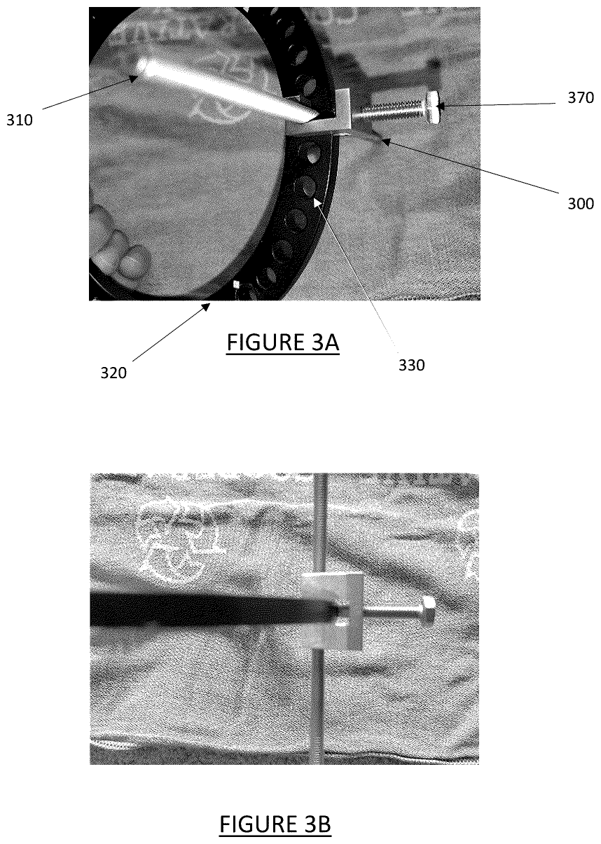

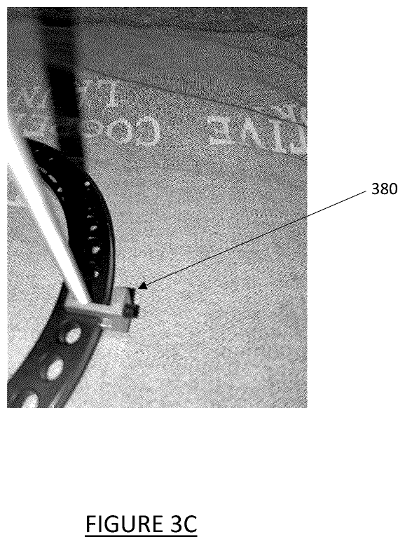

As best seen in FIGS. 3A and 3B, in use the clamping device 300 can be quickly and easily utilized to secure a threaded or unthreaded support rod or strut 310 to a plate member 320, wherein the strut 310 extends through at least one opening 330 in the plate member 320. Once the strut 310 is positioned within a desired opening, the notch of the clamping device 300 can be slid over the plate member 320, and the hook ends of the arms guided over and/or around the strut 310 such that the strut is captured therein. The compression screw 370 can then be rotated and advanced, a distal end of which desirably contacts and compresses against an edge surface of the plate member, drawing the hook ends towards a peripheral edge of the plate member and concurrently moving or “sliding” the captured strut towards a side surface or edge of the opening 330. Once the compression screw 370 has been sufficiently tightened, the hook ends of the arms will desirably immobilize the strut in a desired position and orientation relative to the plate member. With the various surfaces and/or engagements between the strut, plate member and clamping device holding the strut in a rigid position relative to the plate member, which desirably includes prevention of any “toggle” between the strut and the plate member.

If desired, one or more surfaces of the compression device can be textured or roughened to increase the frictional and/or “locking” effect of the device. If desired, surfaces of the strut and/or plate could include corresponding roughened surfaces, desirably contributing to an easily reversed “locking” effect upon tightening of the compression screw. In a similar manner, one or more surfaces of the compression screw, such as the distal tip, could include textured or roughened surfaces.

In various alternative embodiments, some or all of the struts may include unthreaded and/or smooth surfaces while the plate holes may incorporate threaded and/or roughened/textured interior surfaces, or visa-versa. Such an arrangement may be particularly useful to provide temporary support for a missing or removed strut element from a different manufacturer (i.e., where the installed struts and plate holes are threaded, but the temporary support structs are unthreaded), or where different manufactured system components may be combined into a single fixation frame for a variety of reasons.

If desired, the compression screw 370 may be slightly released to allow some relative motion between the strut and the plate member (i.e., allow the rod to “slide” relative to the plate member), without completely releasing the various components, and then subsequently retightened after a desired realignment of the strut and/or plate member has been achieved.

FIG. 3C depicts an alternative embodiment of a clamping device 380 that can be quickly and easily utilized to secure a threaded or unthreaded support rod or strut to a plate member, with a set screw utilized to secure the device in a desired position.

In various embodiments, the disclosed clamping devices can desirably be utilized to quickly and easily connect various plate members and/or struts in a lightweight and durable manner, and also allow the quick and convenient removal and/or realignment of fixation frame components without any need for completely disassembly and/or removal of the entire fixation frame or portions thereof. The disclosed devices can be particularly useful to attach rod to a frame or other object in a nontraditional manner—as compared to existing systems that attach frame elements together with a nut on a threaded rod on top and bottom of the frame. The disclosed clamping devices are desirably designed to provide stabilization of a rod through a plate or structure when the side of the plate or structure allows access.

The various disclosed embodiments provide for frame construction and securement using connecting devices that are easier and far faster than threading nuts onto rods. In existing frame assemblies, if assembly error occurs and a nut is absent on the rod, the user is forced to disassemble the other nuts from the fame element to allow the absent nut to be properly positioned. However, with the present connection systems such activities would not be required. Rather, the disclosed connection systems merely require a user to apply the rod through the plate, and then secure or “hook” the rod to the plate with the disclosed clamp. The disclosed arrangements also allow components of the rod/plate assembly to be changed quickly during a case. For example, a middle plate can be quickly raised or lowered and/or reattached easily, or even be removed from an existing construct without disturbing the remaining of the construct. Longer struts can be utilized along the entire length of the fixation construct without fear that removing one strut would destabilize the remaining construct. Desirably, the disclosed connecting devices can be changed and/or replaced at will to accommodate a different sized plate or structure or different hole locations on the plate or structure. The screw itself can optionally be made more robust for better stabilization as well.

In various embodiments, and depending on the load of the plate, a non-threaded rod and tap through hook may be used to stabilize the construct.

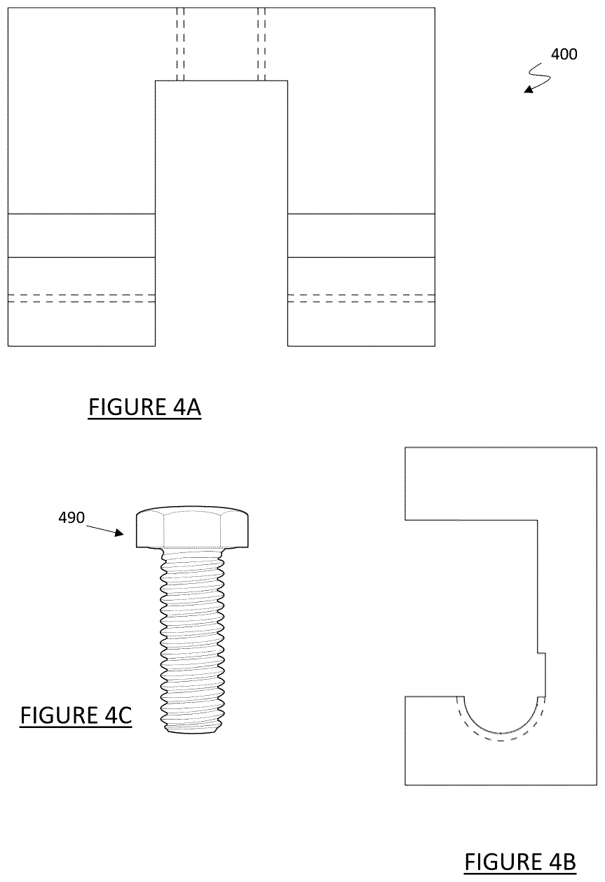

FIGS. 4A and 4B depict front and side plan views of one exemplary embodiment of a connection device 400. FIG. 4C depicts a side view of an exemplary compression bolt 490 which can be utilized with the connection device 400 of FIGS. 4A and 4B.

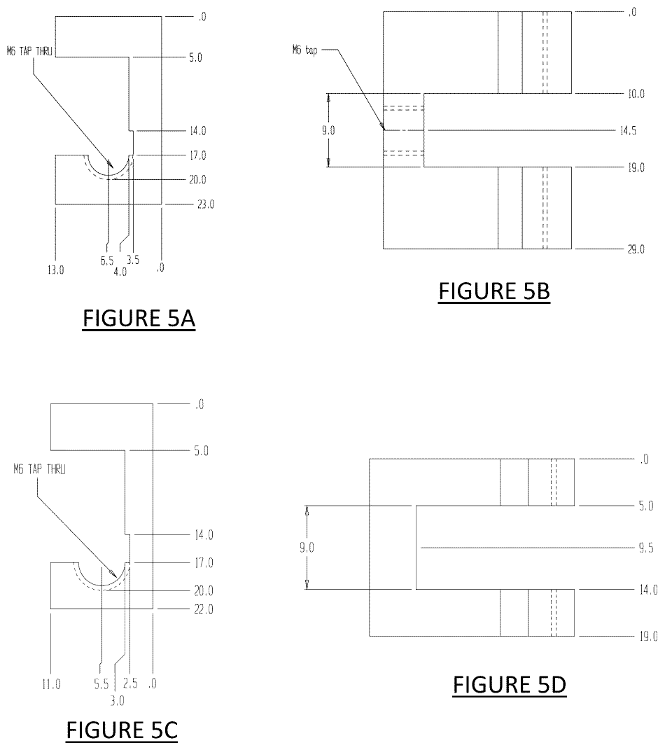

FIGS. 5A and 5B depict side and front views of one exemplary embodiment of a clamping device with various dimensional features shown. FIGS. 5C and 5D depict side and front views of another exemplary embodiment of a clamping device with various representative dimensional features shown. In various alternative embodiments, the various dimensions and/or measurements of the disclosed clamping devices can widely vary, including one or more of the following:

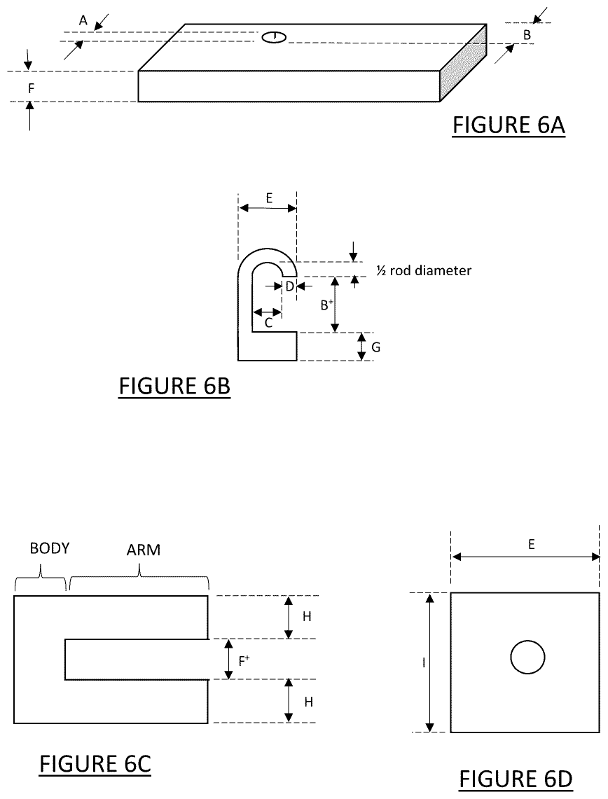

FIGS. 6A through 6D depict a variety of exemplary dimensional features that may be altered for various embodiments of a clamping device, including:

| | |
|---|---|
| A | size of hole in the plate accepting the rod |
| B | size of arm of the retainer with added clearance to get around the rod |
| C | rod size with tap through |
| D | Curved portion of the retention feature - may be about half thickness |
| | of the rod |
| E | rod diameter with tap through - may be rod diameter plus 5 mm in |
| | some embodiments |
| F | thickness of the plate or structure that the top and bottom arms of the |
| | connecting device will “trap” - desirably slightly larger than or equal |
| | to the width of the plate - may be H + H + F, and optionally 1 mm |
| | extra in some embodiments |
| G | body of the connecting device - size depending on strength needed |
| H | thickness of top or bottom arm of the connecting device |
| I | main body of connector - width may be as much as needed for |
| | strength and to accommodate notch width |

One or more of the above listed measurements can vary depending on the size of the rod used and thickness/dimensions of the frame plate or structure to be covered by the arms. In addition, thickness, material selection and strength can vary depending on the load applied to the connecting device. The size of set screw/bolt will vary as well depending on strength needed to hold the connecting device against the plate and or structure. If desired, the set screw/bolt can optionally incorporate a locking component to make loosening of the bolt/set screw harder and/or less likely to occur due to inadvertent contact.

If desired, various modifications can be made to the connection device to allow a single arm device to go between a structure or a plate with a slot.

The disclosed devices may be particularly useful in a variety of situations, including (1) when it may be desirous to change a position or orientation of a support rod or strut to provide a better position for a skinny wire or half pin on the construct, (2) when it is desirous to remove a single rod or strut (or possibly multiple rods and struts) that may prevent easy access to a dressing or underlying anatomy (such as where a rod may have been positioned to protect a dressing or to keep a patient from rubbing an area), wherein the rod may be replaced after the dressing or anatomy is accessed for various reasons, and (3) to allow quick and easy assembly and/or disassembly of a frame or portions thereof for any reason.

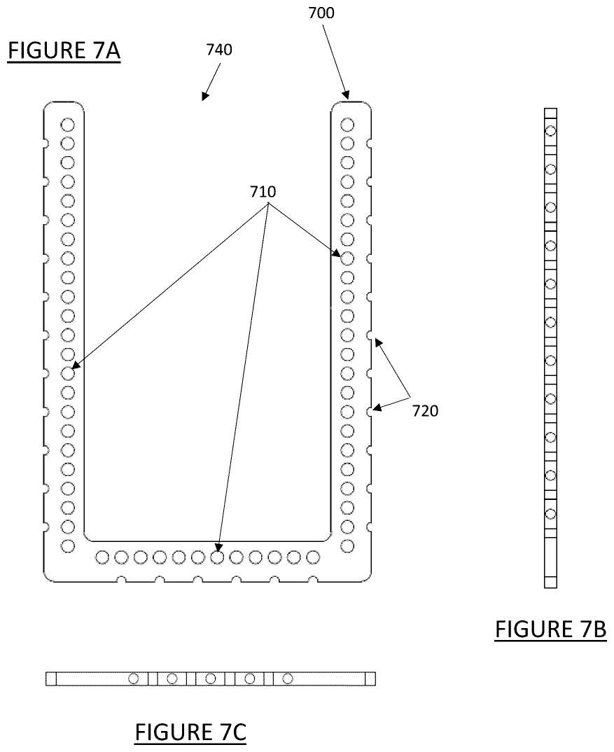

FIGS. 7A through 7C depict plan, bottom and side views of an exemplary fixation plate 700 that may be used with the disclosed invention. In this embodiment, the plate 700 includes a top surface and a bottom surface, with a series of holes or opening 710 extending therethrough. If desired, some or all of the openings 710 may be internally threaded and/or of non-circular shape, although generally the fixation plate holes will desirably be of equal or larger diameter than the outer diameter of the support struts which extend therethrough (which may include holes that are significantly larger than the strut outer diameter, such as holes that may be 1 mm larger in diameter or greater, or 2 mm in diameter or greater, or 3 mm larger in diameter or greater, or 4 mm in diameter or greater, or 5 mm in diameter or greater). The openings 710 may be cylindrical or conical or frustoconical or other cross-sectional shapes as desired. The openings 710 may be of the same or different sizes.

In use, the plate 700 desirably will be provided in a fixation frame kit containing a variety of different sized and/or shaped plates and other frame components and strut components. In use, the plate 700 can be desirably positioned around a limb or extremity (or other anatomy), with an open end 740 of the plate advanced over a treated limb (not shown), with the limb ultimately positioned within a recess of the plate. A support strut or rod (not shown) can be placed through one of the openings, and a connection device such as previously described slid over the plate and rod and the set screw tightened to secure the rod and plate together as shown in FIGS. 3A through 3C.

In at least one alternative embodiment, the disclosed devices may also be utilized to attached struts or connecting rods to externally accessible holes such as edge holes 720 (shown in FIG. 7A). In such an arrangement, the rod can be laterally slid into the edge hole 720, with the connecting device positioned around the plate and engaged with the rod as previously described, such that tightening of the set screw will draw the strut into intimate contact with an inner surface of the edge hole and desirably lock the strut relative to the plate. Such an arrangement could greatly simply placement and/or removal of individual frame members and/or frame levels without requiring any alteration and/or disassembly of any other frame components.

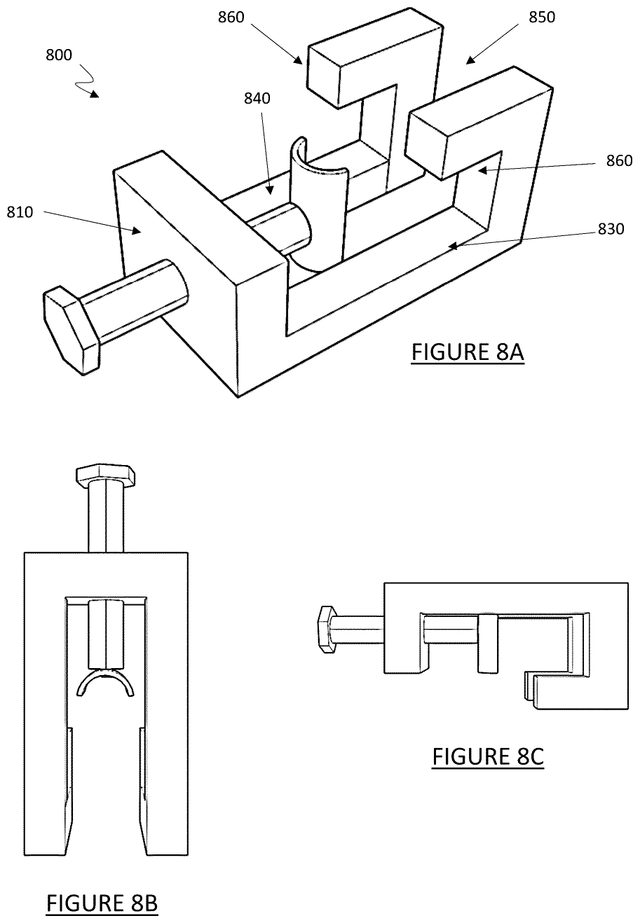

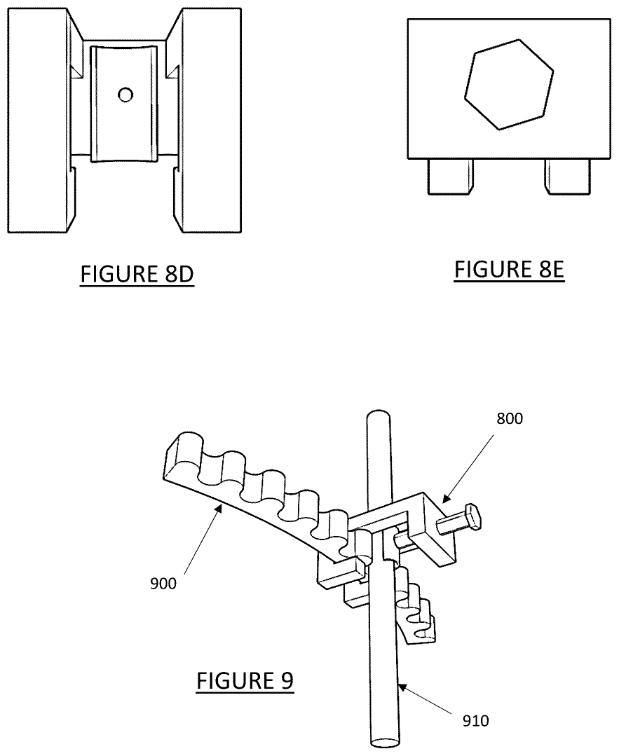

FIGS. 8A through 8E depict another exemplary embodiment of a clamping device 800 to facilitate creation of a support and/or fixation frame that can be particularized for the needs of an individual patient, including the incorporation of various removeable and/or configurable components of the frame that allow the frame to be reconfigured in a variety of ways without requiring removal from and/or loss of support to the patient. In this embodiment, the clamping device 800 includes a base plate 810 having a threaded hole 820 formed therethrough, and a pair of arms 830 and 840 which extend from their proximal ends downward from an edge of the base plate 810. The arms 830 and 840 are desirably separated by a notched section 850, with each arm having a retention portion or hook end 860 positioned at a distal end thereof. An externally threaded set or compression screw 870 can be threaded into and at least partially through the threaded hole 820, with a distal end thereof including a curved contact surface 880 positioned at the distal end via a swivel fitting. If desired, a longitudinal axis of the compression screw will be positioned parallel to at least a portion of arms 830 and 840, although other orientations a contemplated herein.

As best seen in FIG. 9, the clamping device 800 can be utilized to capture a ring member or plate 900 or similar frame structure within the arms, with the compression screw advanced to a desired position, which might include contacting the plate directly (not shown) or alternatively to compress a rod 910 against an indentation in an outer surface of the plate 900, as shown here. For example, if the compression screw of the clamping device is slightly loosened, the rod can be easily be repositioned by sliding relative to the clamping member and/or ring plate without completely releasing and/or disassembling the construct, and then subsequently retightened after a desired realignment of the strut and/or plate member has been achieved.

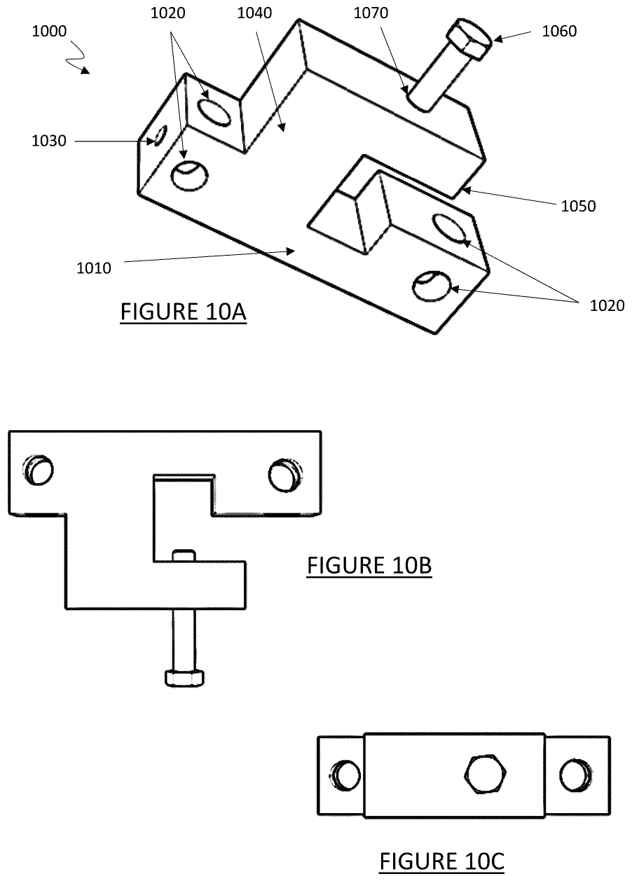

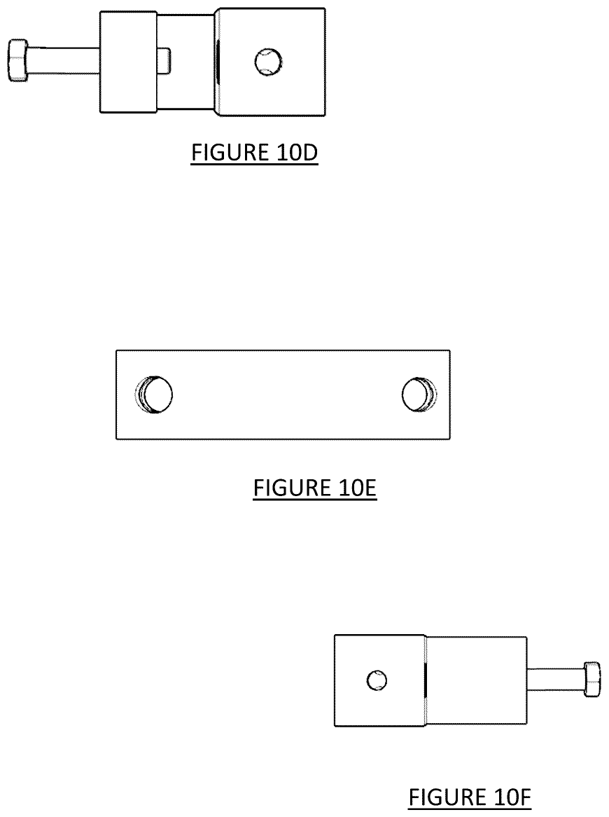

FIGS. 10A through 10F depict another embodiment of a clamping device 1000, which includes a base plate 1010 having openings 1020 to accommodate rods or other attachment devices/components, along with one or more set screw opening 1030 that are desirably sized and configured (and internally threaded) to accommodate a set screw (not shown) or similar locking feature which can be utilized to secure a rod or similar support structure to the device (e.g., within the openings). An arm 1040 can extend from the base plate, with the arm including an overlapping portion 1050 positioned at a distal end thereof. An externally threaded set or compression screw 1060 can be threaded into and at least partially through a threaded hole 1070 formed in the arm 1040.

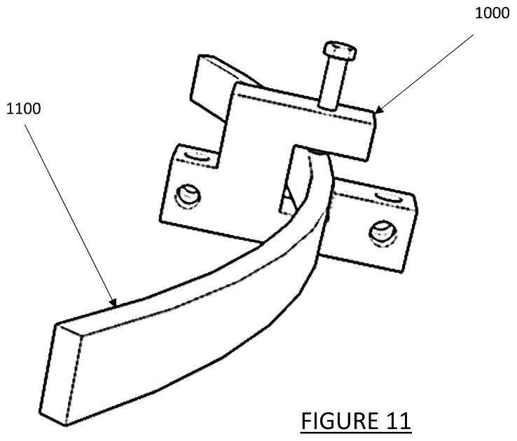

FIG. 11 depicts the embodiment of FIGS. 10A through 10D when connected to a curved frame member 1100, wherein the compression screw 1060 in tightened into contact with and compresses the member 1100. Although not depicted in this figure, one or more rods can extend through various openings in the device 1000 to facilitate securement. If desired, the compression screw of the device can be loosened and/or tightened in a desired manner, as well as loosening or tightening of the various set screws to allow adjustment of rods therein.

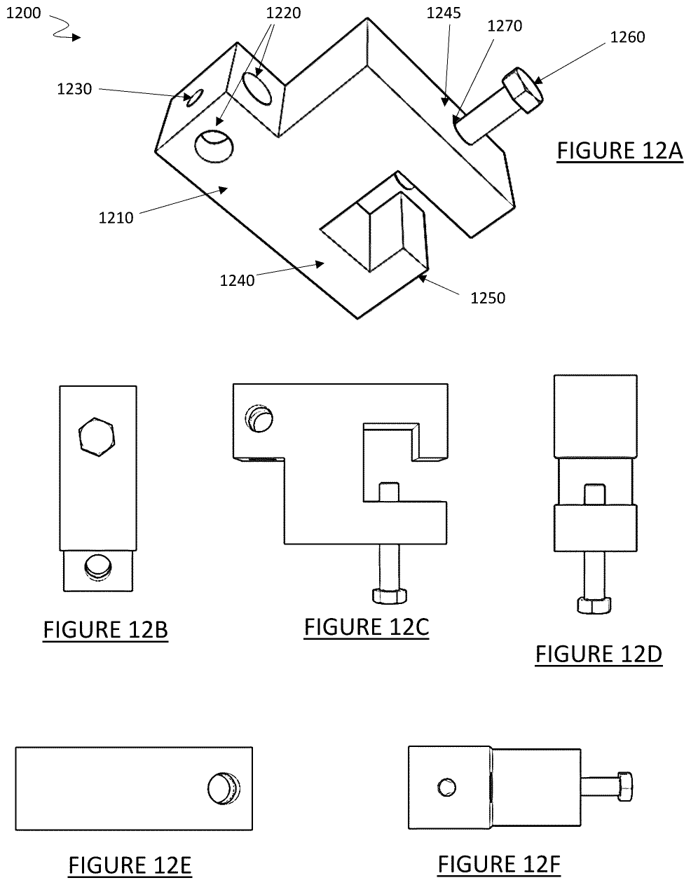

FIGS. 12A through 12F depict another embodiment of a clamping device 1200, which includes a base plate 1210 having one or more openings 1220 to accommodate rods or other attachment devices/components, along with a set screw opening 1230 that is desirably sized and configured (and optionally internally threaded) to accommodate a set screw (not shown) or similar locking feature, which can be utilized to secure a rod or similar support structure to the device (e.g., within the openings). First and second arms 1240 and 1245 can extend from the base plate, with the arm including a retention portion or hook end 1250 positioned at a distal end thereof. An externally threaded set or compression screw 1260 can be threaded into and at least partially through a threaded hole 1270 formed in the arm 1245.

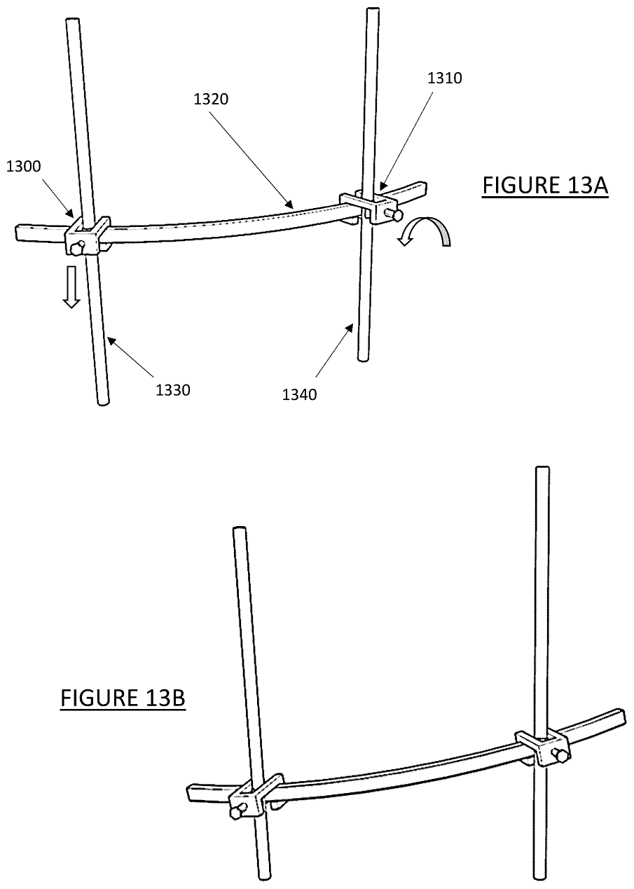

FIGS. 13A and 13B depict a pair of clamping devices connecting to a curved support ring and a pair of threaded or unthreaded support rods, where the clamping devices and support ring can be selectively repositioned on the rods. As shown, the clamping devices 1300 and 1310 of FIGS. 8A through 18F are connected a curved frame member 1320 and a first rod 1330 and a second rod 1340. The various features of the clamping devices 1300 and 1310 allow these devices to be installed, tightened, loosened and/or repositioned relative to other components without requiring disassembly and/or removal of an existing extremity support frame. In many cases, these elements can be attached to an existing support frame, which can reinforce the existing frame and/or allow some or all of the frame to be disassembled and/or rebuilt to accomplish various objectives. Similarly, various design features of the disclosed devices allow them to be reoriented and/or repositioned while still providing varying degrees of support for the patient's anatomy (for example, the component rotation, displacement and/or other movement from FIG. 13A to FIG. 13B).

In various embodiments, the devices and systems disclosed herein can facilitate creation of a support and/or fixation frame that can be particularized for the needs of an individual patient, including the incorporation of various removeable and/or configurable components of the frame that allow the frame to be reconfigured in a variety of ways without significantly affecting the strength and/or stability of the frame or requiring removal from the patient. Whenever necessary and/or desired, reconfiguring of the frame can allow a surgeon, physician and/or other caregiver to access virtually any area of the patient's anatomy to inspect and/or treat wounds, other injuries and/or an intended surgical site, as well as to allow access to many anatomical surfaces and/or other areas of the patient for additional treatments up to and including surgical procedures on the supported anatomy.

In various embodiments, the disclosed plates can include full plates and/or partial plates, such as straight plates, angle or half plates and/or three-quarter plates, as well as U-shaped plates (including straight and/or curved plates and/or portions thereof. The plates will desirably include a variety of vertical and/or transverse holes for coupling additional components to the plates. Each plate can desirably be removably and/or slidably coupled to adjacent frame structures with one or more rods or struts that can, for example, be configured to provide load-bearing support. A plate can be attached to a strut with a connection device, or the rod may include features (i.e., external threads) that can engage with internal structures (i.e., internal threading) in some or all of the holes or depressions. The struts could similarly be further coupled together by various fixation components or coupling members (i.e. trusses) which can, e.g., increase the stability of the device.

During use, if a frame component obstructs a portion of the extremity or other anatomical feature in an undesired manner, the physician can loosen one or more individual connection devices of the frame, and reposition the rods and/or other components to a new location, where the connection device can be retightened in the new location without compromising the fixation or strength of the frame during such manipulation—thereby allowing the physician to access the wound for treatment, even allowing the placement of a negative pressure wound therapy device (i.e., “wound vac”), if desired. Similarly, if fixation pins or other bony attachment points (or frame components) require manipulation or repositioning from a first to a second position, the frame members and/or fixation pins can be moved in a similar manner without affecting other fixation provided by the frame during such movement. In this way, portions of the frame and/or other support structures can be modified without comprising or affecting the support and fixation concurrently provided by the remainder of the frame, thereby allowing unfettered access virtually all of the patient's anatomy in a desired manner.

While the various embodiments described herein may depict straightened, squared and/or rectangular (i.e., more angular) designs for fixation plates and/or other components, it should be understood that virtually any polygonal shape (and/or curved, oval and/or circular shapes) as well as combinations thereof could be useful for the various plates and/or other components, including triangular, pentagonal, hexagonal, septagonal, octagonal and/or rounded shapes, if desired. In some embodiments, square and/or rectangular system components may be easier to upsize and/or accommodate a variety of modular components, and may also be less expensive to manufacture. Moreover, the use of flat, angular and/or squared components can significantly reduce the require diameter of the fixation frame and/or allow the fixation frame to lay flat upon the one or more sides, which can be much more comfortable for the physician and/or wearer. In addition, there is typically more room underneath a squared frame between the frame and skin surface to accomplish dressing changes, as well as more space to modify dressings and/or add to dressings, as well as accommodate other items and/or equipment. This may be particularly useful for patient with obese legs or large calves, wherein the physician can shift the frame posterior (unlike rounded frames which are typically centered on the limb axis). Moreover, a squared frame design facilitates the use of offset rods and/or rod connectors, whereas offset of a rounded system might potentially tilt the components of a round frame design in an unexpected manner.

In addition, the incorporation of flattened and/or angular plate components allows the use of a wide variety of clamp designs and/or other components to assemble the fixation frame—including the use of components of different sizes and/or shapes in a single construct. Various connector designs can be utilized on a single level and/or frame member, which allows for many different ways to bolt or otherwise connect different size components together, which is not easily accomplished with many existing fixation systems.

If desired, the disclosed frame designs can optionally accommodate curved components such as c-shaped and/or u-shaped plates as a main level component, with opposing flat and/or C/U-shaped components to optionally close off the “box” on each level. In fact, various designs could incorporate rounded and/or curved sections, including rounded frame members (i.e., to create D-shaped frame members, for example) and/or curved support rods between frame sections. In a similar manner, the various components described herein could potentially accommodate and/or attach to components from other fixation systems, including rounded systems, if desired.

The various fixation frame components, including squared frame components, can be utilized to better “space out” the connecting rods and/or struts (including the use of rods in corners of squared frame sections), allowing a physician to change rod locations in office and/or much easier to add levels in the office or in surgery on a frame already on a patient. Moreover, the disclosed embodiments are faster to assemble and disassemble than existing designs, allow faster dressing changes, and these design also require significantly fewer parts.

In addition to easy of assembly, the disclosed frame designs desirably include a variety of features to allow frame components to be added and/or removed from the frame with little disruption to adjacent frame members. For example, current frame designs typically include rods and numerous nuts on inside surfaces of frame, with nuts on the ends of rods. In order to disassembly and/or modify these frame components, a surgeon or physician must unscrew all the nuts at both the top and bottom of a rod before rod removal can be accomplished. Moreover, if a nut or other component goes missing, it may be necessary to rebuild the entire frame to accommodate the missing components, and it is typically difficult to add frame sections in an out-patient environment.

The presently disclosed embodiments will desirably obviate the need for multiple assistants during frame placement and/or assembly. During a current surgical procedure, a surgeon will often utilize an assistant or folded and stacked towels, etc., to hold the patient's leg in a desired position and/or orientation within the frame to facilitate assembly. However, with the currently disclosed designs, the physician can optionally utilize the easily removable and/or replaceable connection devices to alter the frame as desired, including the ability to reinforce sections of the frame and/or move rods and/or other components to allow pin positions and/or other anatomical features to be exposed in a desired manner.

One or more structures as described herein may be provided in the form of a kit. A kit may be assembled for portability, facilitating use in a surgical setting, and the like. A kit can typically include various components of an orthopedic fixation system of the present invention, and the orthopedic fixation system may be provided in a fully assembled, partially assembled, or non-assembled configuration. As indicated, a device of the present invention may be configured or of a designed such that one or more components of the fracture reduction system have a limited or single use, or are replaceable. A kit may include pre-sterilized components or device(s), as well as sterilized packaging. The components of the present invention may be sterilized (and will generally be sterilizable) by any of the well-known sterilization techniques, depending on the type of material. Suitable sterilization techniques include heat sterilization, radiation sterilization, chemical/gas sterilization, and the like.

In yet other embodiments, the present invention provides methods of using the devices and assemblies described herein. In an exemplary embodiment, the present invention provides a method of using an orthopedic fixation system can include modifying the fixation device in an outpatient or transportation setting while the patient's bone is immobilized with the fixation device.

Structures, devices, and assemblies of the present invention should not be limited to any particular construction materials or compositions. Materials and compositions of the invention can include any variety of metals, alloys, polymers, and the like, alone or in combination, that are commonly used or generally suitable for use in medical or surgical applications. Devices and components thereof may be made from conventional non-absorbable, biocompatible materials including stainless steel, titanium, alloys thereof, polymers, composites and the like and equivalents thereof. The use of lighter materials such as high strength metals, plastics and/or ceramics will desirably allow a square frame to have equivalent and/or lighter weight than a corresponding rounded frame and its components.

The specific dimensions of any of the orthopedic fixation systems, assemblies, and components thereof, of the present invention can be readily varied depending upon the intended application, as will be apparent to those of skill in the art in view of the disclosure herein. Moreover, it is understood that the examples and embodiments described herein are for illustrative purposes only and that various modifications or changes in light thereof may be suggested to persons skilled in the art and are included within the spirit and purview of this application and scope of the appended claims. Numerous different combinations of embodiments described herein are possible, and such combinations are considered part of the present invention. In addition, all features discussed in connection with any one embodiment herein can be readily adapted for use in other embodiments herein. The use of different terms or reference numerals for similar features in different embodiments does not necessarily imply differences other than those which may be expressly set forth. Accordingly, the present invention is intended to be described solely by reference to the appended claims, and not limited to the preferred embodiments disclosed herein.

The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

General

Many of the aspects and advantages of the present invention may be more clearly understood and appreciated by reference to the accompanying drawings. The accompanying drawings are incorporated herein and form a part of the specification, illustrating embodiments of the present invention and together with the description, disclose the principles of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein.

The various headings and titles used herein are for the convenience of the reader, and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A method of securing a first fixation plate to a support strut in a fixation frame assembly, comprising the steps of:
positioning the support strut through an opening which extends through the first fixation plate,
positioning a connection device around at least an edge surface portion of the first fixation plate such that a first portion of the connection device is positioned around a first portion of the support strut proximate to a first surface of the fixation plate and a second portion of the connection device is positioned around a second portion of the support strut proximate to a second surface of the fixation plate, and
advancing a set screw which extends into the connection device, the set screw contacting the fixation plate and drawing the support strut into intimate contact with an inner edge wall of the opening extending through the fixation plate such that the support strut is immobilized relative to the fixation plate.

2. The method of claim 1, wherein the first surface of the fixation plate comprises a top surface of the fixation plate and the second surface of the fixation plate comprises a bottom surface of the fixation plate, and the opening extends through the first fixation plate from the top surface to the bottom surface.

3. The method of claim 1, wherein the connection device includes a roughened or textured surface which engages with a corresponding roughened or textured surface of the support strut.

4. The method of claim 1, wherein the connection device includes a roughened or textured surface which engages with a corresponding roughened or textured surface of the fixation plate.

5. The method of claim 1, wherein the set screw includes a roughened or textured distal tip.

6. The method of claim 1, further comprising the step of positioning the support strut through a second opening which extends through a second fixation plate,
positioning a second connection device around at least an edge surface portion of the second fixation plate such that a first portion of the second connection device is positioned around a second portion of the support strut proximate to a first surface of the second fixation plate and a second portion of the second connection device is positioned around a second portion of the support strut proximate to a second surface of the second fixation plate, and
advancing a second set screw which extends into the second connection device, the second set screw contacting the second fixation plate and drawing the support strut into intimate contact with a second inner edge wall of the opening extending through the second fixation plate such that the support strut is immobilized relative to the second fixation plate.
